(19) United States
(12) Patent Application Publication
(10) Pub. No.: US 2024/0181253 A1
(43) Pub. Date: Jun. 6, 2024

(54) MIGRAINE TREATMENT DEVICE

(71) Applicant: HI-DOW IPHC, INC., St. Louis, MO (US)

(72) Inventors: Luying MENG, Hefei (CN); Yang CHEN, Hefei (CN); Bin DU, Hefei (CN)

(73) Assignee: HI-DOW IPHC, INC., St. Louis, MO (US)

(21) Appl. No.: 18/131,284

(22) Filed: Apr. 5, 2023

(30) Foreign Application Priority Data
Dec. 1, 2022 (CN) 202223214393.5

(51) Int. Cl. A61N 1/36; A61F 7/00; A61H 23/02; A61M 21/02; A61N 1/04; A61N 5/06

(52) U.S. Cl. CPC A61N 1/36021; A61F 7/007; A61H 23/0254; A61M 21/02; A61N 1/0456; A61N 1/0484; A61N 5/0613; A61F 2007/0075; A61H 2201/0192; A61H 2201/0207; A61H 2201/0285; A61H 2201/10; A61H 2201/1604; A61H 2201/5048; A61M 2021/0027; A61N 2005/0626; A61N 2005/0648; A61N 2005/0652; A61N 2005/0663

(57) ABSTRACT

A migraine treatment device in the form of an eye mask is provided. The migraine treatment device includes a main body in the form of an eye mask structure. Fixed therapeutic electrode pads are provided on the inner side of the main body near the top. The inside of the main body is equipped with a green light-emitting board, which is evenly arranged with green LED lights on both sides. Additionally, the inside of the main body has a light-transmitting component that is compatible with the green light-emitting board and a lithium battery for supplying power to the migraine treatment device.

### CROSS-REFERENCE TO RELATED APPLICATION

This application is filed based upon and claims priority to Chinese patent application No. 202223214393.5, filed on Dec. 1, 2022, the entire content of which is incorporated herein by reference for all purposes.

### TECHNICAL FIELD

The disclosure generally relates, but not limited, to the technical field of migraine treatment devices, and more particularly, to a migraine treatment device in the form of an eye mask.

### BACKGROUND

Migraine often begins in childhood and adolescence, reaches a peak incidence in middle age, is more common in women, and often has a genetic background. It is mainly characterized by severe, pulsating headaches that occur intermittently, often lasting from several hours to 72 hours on each occasion. During an attack, there is often nausea, vomiting, and sensitivity to light, sound, or normal activities, which can exacerbate symptoms, while a quiet environment and rest can provide relief. The condition can be controlled through lifestyle adjustments and medication.

During a migraine attack, various pain-relieving medications are used to terminate the pain, while in between attacks, the main objective is to prevent future migraines. However, long-term use of medication can have side effects and harm the body.

Research has shown that exposing migraine sufferers to LED green light for green light therapy can reduce the number of migraine days per month, the frequency of migraine attacks, and the duration and intensity of headaches, with no side effects. Additionally, using Transcutaneous Electrical Nerve Stimulation (TENS) technology to treat the trigeminal nerve in the forehead with electrical pulses can alleviate migraines.

### SUMMARY

The present disclosure provides a migraine treatment device that may have an eye mask structure.

According to a first aspect of the present disclosure, a migraine treatment device is provided. The device may include a therapeutic device main body having a structure of an eye mask, where a fixed therapeutic electrode pad is placed on an upper inner side of the therapeutic device main body. Additionally, a green light emitting board may be placed inside the therapeutic device main body, where green light emitting diode (LED) lights may be evenly arranged on both sides of the green light emitting board. Furthermore, a light transmitting component may be placed inside the therapeutic device main body, where the light transmitting component may be adapted to the green light emitting board. Moreover, the migraine treatment device may include a lithium battery that supplies power to the therapeutic device main body.

It is to be understood that the above general descriptions and detailed descriptions below are only exemplary and explanatory and not intended to limit the disclosure.

### BRIEF DESCRIPTION OF THE DRAWINGS

The accompanying drawings, which are incorporated in and constitute a part of this specification, illustrate embodiments consistent with the disclosure and, together with the description, serve to explain the principles of the disclosure.

FIG. 1 shows a schematic diagram of an overall structure of a migraine treatment device according to an example of the present disclosure.

FIG. 2 is a schematic diagram illustrating connecting the therapeutic device main body and mobile therapeutic electrode pads according to an example of the present disclosure.

FIG. 3 is a schematic diagram illustrating connecting the therapeutic device main body and ear clip wire according to an example of the present disclosure.

FIG. 4 is a schematic diagram illustrating the therapeutic device main body in a folded manner according to an example of the present disclosure.

FIG. 5 is an exploded view illustrating the overall structure of the therapeutic device main body according to an example of the present disclosure.

FIG. 6 is a schematic diagram illustrating the internal structure of the therapy device main body behind the front mask according to an example of the present disclosure.

### DETAILED DESCRIPTION

Reference will now be made in detail to exemplary embodiments, examples of which are illustrated in the accompanying drawings. The following description refers to the accompanying drawings in which the same numbers in different drawings represent the same or similar elements unless otherwise represented. The implementations set forth in the following description of examples do not represent all implementations consistent with the disclosure. Instead, they are merely examples of apparatuses and methods consistent with aspects related to the disclosure as recited in the appended claims.

Reference throughout this specification to “one embodiment,” “an embodiment,” “an example,” “some embodiments,” “some examples,” or similar language means that a particular feature, structure, or characteristic described is included in at least one embodiment or example. Features, structures, elements, or characteristics described in connection with one or some embodiments are also applicable to other embodiments, unless expressly specified otherwise.

The terms “module,” “sub-module,” “circuit,” “sub-circuit,” “circuitry,” “sub-circuitry,” “unit,” or “sub-unit” may include memory (shared, dedicated, or group) that stores code or instructions that can be executed by one or more processors. A module may include one or more circuits with or without stored code or instructions. The module or circuit may include one or more components that are directly or indirectly connected. These components may or may not be physically attached to, or located adjacent to, one another.

For making it convenient for those skilled in the art to understand, multiple implementation modes are listed in the embodiments of the disclosure to describe the technical solutions of the embodiments of the disclosure clearly. Of course, those skilled in the art can understand that multiple embodiments provided in the embodiments of the disclosure can be executed independently, or can be combined with methods of the other embodiments in the embodiments of the disclosure for execution together, or may be executed independently or after combined with some methods in other related technologies. No limits are made thereto in the embodiments of the disclosure.

The present disclosure provides a migraine treatment device that may be in the form of an eye mask, which includes LED green light, TENS technology, heat therapy, music, and vibration massage functions. The migraine treatment device may provide green light therapy and electrical pulse therapy, supplemented by heat therapy, music, and vibration massage to relieve migraine symptoms.

To achieve the purpose above, the present disclosure provides following technical solutions: a migraine treatment device in the form of an eye mask, including a therapeutic device main body with an eye mask structure. The therapeutic device main body may have fixed therapeutic electrode pads located near the upper inner side of the therapeutic device main body. The internal part of the therapeutic device main body may be equipped with a green light emitting board, with green LED lights arranged evenly on both sides of the green light emitting board. Additionally, the internal part of the therapeutic device main body may include a light transmitting component that is compatible with the green light emitting board and a lithium battery for powering the therapeutic device main body.

In some examples, movable therapeutic electrode pads and ear clip wires may be provided. additionally, electrode connecting wires may be connected with the movable therapeutic electrode pads. The external part of the therapeutic device main unit may be equipped with output interfaces that are compatible with the electrode connecting wires and the ear clip wires.

In some examples, the therapeutic device main body may have a foldable structure. The overall casing of the therapeutic device main body may include a first casing component, two second casing components, and two third casing components that are sequentially arranged from an outer side to an inner side. Furthermore, the two second casing components may be respectively pivotally connected to both sides of the first casing component and the two third casing components may be respectively fixedly connected to the inner sides of the two second casing components.

In some examples, a main mask may be placed at an outer side of a front end of the therapeutic device main body.

In some examples, the internal part of the therapeutic device main body may be equipped with a microphone and a speaker. The external/outer wall of the therapeutic device main body may be equipped with a signal receiving hole, a charging port, and one or more buttons.

In some examples, a first foam component, a second foam component, a third foam component, and an elastic fabric may be successively placed on an inner side of the overall casing of the therapeutic device main body, and a female buckle may be placed on an outer side of the elastic fabric, a plurality of through holes may be provided on the second foam component, and a vibration motor may be provided in each through hole.

In some examples, a heating device and a thermistor that is adapted to/compatible with the heating device may be displaced inside the therapeutic device main body, and the heating device may be located between the second foam component and the third foam component.

In some examples, straps and adjustment devices that are adapted to the straps may be displaced on both sides of the therapeutic device main body.

The migraine treatment device provided in the present disclosure uses fixed therapeutic electrode pads connected to the therapeutic device main body to transmit electric pulses to the forehead area of the user to treat migraines. Additionally, the migraine treatment device has a built-in green light emitting board that emits green light through a translucent part of the columnar structure for the user's eyes to perceive. As a result, the provided migraine treatment device reduces the number of days, frequency, duration, and intensity of migraines per month without side effects. Furthermore, the therapeutic device main body may include a face mask to increase the effectiveness of the green light therapy by reducing the transparency of the mask and blocking external light interference.

The therapeutic device main body of the provided eye mask-style migraine treatment device provides LED green light, TENS technology, heat therapy, music, and vibration massage functions. The device uses a flexible eye mask-style structure to cover the area around the user's eyes, blocking out ambient light. The straps and adjustment device provide good coverage and versatility, while also making it convenient to store.

Furthermore, this eye mask-style migraine treatment device uses a three-layer foam structure, with a vibration motor located between the first and third foam components. The third foam component is thinner, allowing the vibration motor to create raised features on the elastic fabric, thus effectively improving the vibration massage effect.

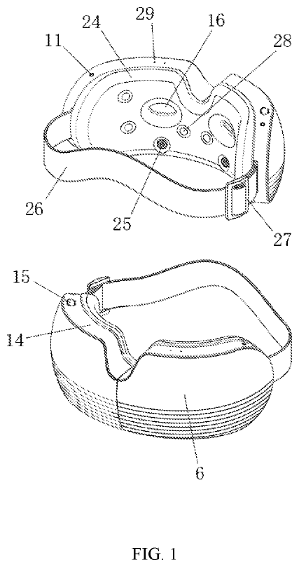

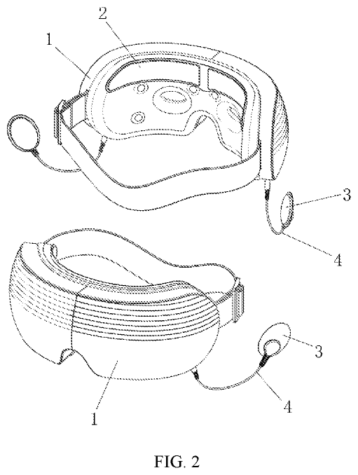

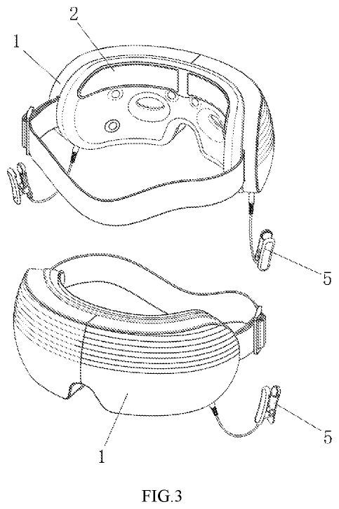

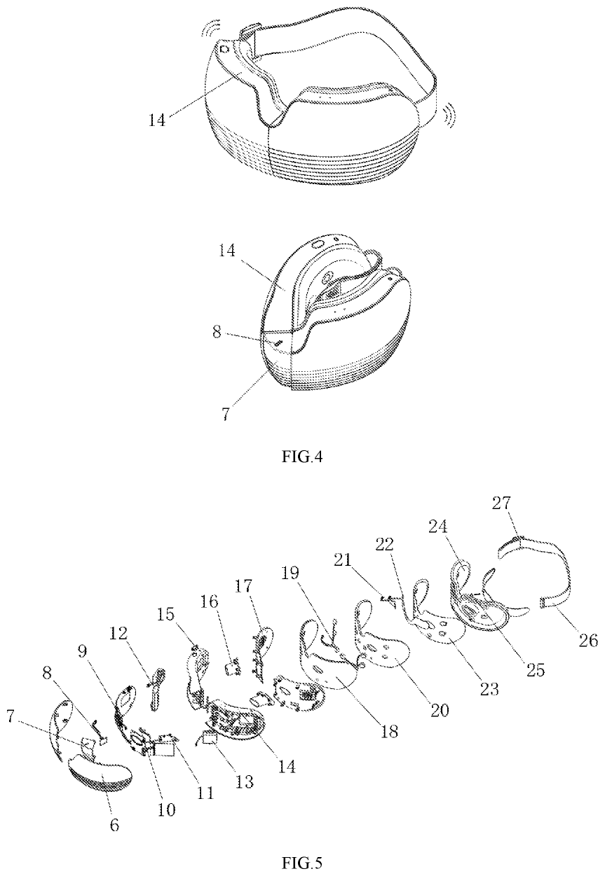

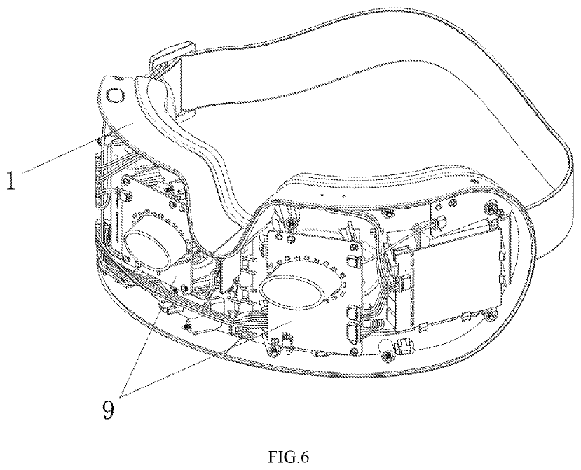

FIGS. 1-5 illustrate the structure of the migraine treatment device in different perspectives. FIG. 1 shows a schematic diagram of an overall structure of a migraine treatment device according to an example of the present disclosure. FIG. 2 is a schematic diagram illustrating connecting the therapeutic device main body and mobile therapeutic electrode pads according to an example of the present disclosure. FIG. 3 is a schematic diagram illustrating connecting the therapeutic device main body and ear clip wire according to an example of the present disclosure. FIG. 4 is a schematic diagram illustrating the therapeutic device main body in a folded manner according to an example of the present disclosure. FIG. 5 is an exploded view illustrating the overall structure of the therapeutic device main body according to an example of the present disclosure.

As shown in FIGS. 1-5, the therapeutic device main body 1 provides functions including LED green light, TENS technology, heat therapy, music, and vibration massage. The therapeutic device main body 1 may be voice-controlled based on online intelligent speech recognition control chip technology, remotely controlled based on wireless control technology using a remote control, and controlled using IOS/Android mobile phone APP based on Bluetooth technology.

In some examples, one or more fixed therapeutic electrode pads may be arranged near the upper inside of the therapeutic device main body 1. As shown in FIG. 1, two fixed therapeutic electrode pads 2 are placed on the upper inner side of the therapeutic device main body 1. The fixed therapeutic electrode pads 2 may be connected to the therapeutic device main body 1 to be used in conjunction with the therapeutic device main body 1 to transmit electric pulses to the forehead to treat migraines.

In some examples, green light emitting boards 9 may be provided inside the therapeutic device main body 1. Green light LED lights may be placed evenly on both sides of each green light emitting board 9, as shown in FIG. 5. In some examples, the wavelength of the green light LED lights may be 525 nm.

In some examples, light transmitting components 16 that are compatible with the green light emitting board 9 and a lithium battery 12 for powering the therapeutic device main body 1 may also be placed inside the therapeutic device main body 1, as shown in FIG. 5. The light transmitting components 16 may transmit the LED green lights emitted from the green light emitting board 9, while the lithium battery 12 may provide power to the entire migraine treatment device.

In some examples, the migraine treatment device may further include movable therapy electrode pads 3 and ear clip wires 5, as shown in FIGS. 2-3. Each movable therapy electrode pad 3 may be equipped with an electrode connection wire 4 to connect the movable therapy electrode pad 3 to the therapeutic device main body 1 to transmit electric pulses. The ear clip wire 5 may connect the therapeutic device main body 1 and transmit electric pulses. An output interface 11, as shown in FIG. 5, that is compatible with the electrode connection wire 4 and the ear clip wire 5 may be provided outside of the therapeutic device main body 1, and the electrode connection wire 4 and the ear clip wire 5 may be inserted into the output interface 11 as needed, allowing the movable therapy electrode sheet 3 to be attached to the trigeminal nerves that the fixed therapy electrode pads 2 cannot cover, or to wear the ear clip wire 5.

In some examples, the therapeutic device main body 1 may have a foldable structure, and the overall casing of the therapeutic device main body 1 may include a first casing component 7, two second casing components 14, and two third casing components 17 respectively from the outside to the inside in order, as show in FIGS. 3-5. The two second casing components 14 are respectively movably and pivotally connected to both sides of the first casing component 7. For example, the two second casing components 14 may be hinged to the both sides of the first casing component 7. Furthermore, the two third casing components 17 are respectively fixedly connected to the inner side of the two second casing components 14, thus making the therapeutic device easily foldable for storage.

In some examples, the outer side of the front end of the therapeutic device main body 1 may be provided with a main mask 6, which can be made of glass or plastic material and uses electrochromic technology to achieve autonomous changes in mask transparency. By changing the transparency of the mask to a visible degree, the migraine treatment device can be controlled using a remote control or IOS/Android mobile phone APP when the main body is worn. By reducing the transparency of the mask to reduce the impact of external light, the effect of LED green light can be enhanced.

In some examples, a microphone 10 and a speaker 13 may be included within the therapeutic device main body 1. The microphone 10 may be used to receive voice signals and convert them into electrical signals to achieve voice control function. The speaker 13 may convert electrical signals into sound signals for playing voice feedback and music.

In some examples, the outer wall of the therapeutic device main body 1 may be equipped with a signal receiving hole 29, a charging port 8, and a button 15. As shown in FIGS. 4-5, the outer wall may include the second casing components 14. The signal receiving hole 29 may receive signals from the microphone 10, and the charging port 8 may be a TYPE-C interface for charging the therapeutic device main body.

In some examples, the inside of the overall casing of the therapeutic device main body 1 may be equipped with a first foam component 18, a second foam component 20, a third foam component 23, and an elastic fabric 24. The second foam component 20 may having several through-holes, and a vibration motor 19 may be installed in each through-hole to achieve a vibrating massage effect. The outer surface of the elastic fabric 24 may have raised features 28, which are slightly raised features formed by the vibration motor 19 on the surface of the elastic fabric 24, thereby improving the effect of facial vibration massage.

In some examples, the therapeutic device main body 1 may be equipped with a heating device 22 and a thermistor 21 adapted to the heating device 22. As shown in FIG. 5, the heating device 22 is located between the second foam component 20 and the third foam component 23 and has a heating effect. Effective heating can be achieved using resistance wire, graphene film, heating film, etc. The thermistor 21 may be used to measure the temperature of the heating device 22.

In some examples, the elastic fabric 24 may be used to wrap around the third casing component 17, the first foam component 18, the vibration motor 19, the second foam component 20, the thermistor 21, the heating device 22, and the third foam component 23 to achieve wrapping and buffering effects. The elastic fabric 24 may also cover the area around the user's eyes and blocks ambient light.

In some examples, the outer side of the elastic fabric 24 may be equipped with a female buckle 25, which is used to connect to the fixed therapeutic electrode pads 2 and transmit electric pulses to the forehead of the user. The two sides of the therapeutic device main body 1 may also be equipped with straps 26 and adjustment devices 27 adapted to the straps 26. The straps 26 may be made of elastic materials, and the adjustment devices 27 may be used in conjunction with the straps 26 to make the therapeutic device main body 1 adaptable to different head circumferences. Since the therapeutic device main body 1 uses a foldable eye mask structure, with the use of straps 26 and adjustment devices 27, it has good wrapping performance. Therefore, the fixed therapeutic electrode pads 2 may be a gel electrode with adhesive properties or a non-adhesive electrode such as conductive silicone rubber or silver fiber fabric.

In some examples, when the user is using the migraine treatment device, the user may press and hold the button 15 to turn it on, and the therapeutic device main body 1 will emit a startup voice prompt. At this time, the user may control the therapeutic device main body 1 through voice control/remote control/mobile APP. The user may also control the electric pulse mode (treatment mode/prevention mode), pause/continue the electric pulse, adjust the electric pulse intensity, turn on/off the heating, adjust the temperature, turn on/off the vibration massage, turn on/off the LED green light, adjust the transparency of the mask, and turn it on/off. After use, the migraine treatment device can be automatically shut down.

According to the examples in the present disclosure, the fixed therapeutic electrode pads are connected to the therapeutic device main body and are used in conjunction with it to transmit electrical pulses that act on the trigeminal nerves in the head to treat migraines. The device also has a built-in green light-emitting board, and the green lights pass through the light-transmitting component in the form of a columnar structure, which is perceived by the user's eyes, reducing the number of migraine days, frequency of migraine attacks, duration, and intensity of headaches per month, without side effects. Additionally, a face mask is provided on the therapeutic device main body to enhance the effectiveness of the green light therapy.

Other implementation solutions of the disclosure will be apparent to those skilled in the art from consideration of the specification and practice of the disclosure. This application is intended to cover any variations, uses, or adaptations of the disclosure following the general principles thereof and including such departures from the disclosure as come within known or customary practice in the art. It is intended that the specification and examples be considered as exemplary only, with a true scope and spirit of the disclosure being indicated by the following claims.

It will be appreciated that the disclosure is not limited to the exact construction that has been described above and illustrated in the accompanying drawings, and that various modifications and changes may be made without departing from the scope thereof. It is intended that the scope of the disclosure only be limited by the appended claims.

What is claims is:

1. A migraine treatment device, comprising:
a therapeutic device main body, wherein a fixed therapeutic electrode pad is placed on an upper inner side of the therapeutic device main body,
a green light emitting board placed inside the therapeutic device main body, wherein green light emitting diode (LED) lights are evenly arranged on both sides of the green light emitting board,
a light transmitting component placed inside the therapeutic device main body, wherein the light transmitting component is adapted to the green light emitting board, and
a lithium battery that supplies power to the therapeutic device main body.

2. The migraine treatment device of claim 1, further comprising a movable therapeutic electrode pad and an ear clip wire, wherein an electrode connection wire is connected to the movable therapeutic electrode pad, and an output interface that is adapted to the electrode connection wire and the ear clip wire is placed outside the therapeutic device main body.

3. The migraine treatment device of claim 1, wherein the therapeutic device main body has a foldable structure, and an overall casing of the therapeutic device main body comprises a first casing component, two second casing components, and two third casing components that are sequentially arranged from an outer side to an inner side, the two second casing components are respectively pivotally connected to two sides of the first casing component, and the two third casing components are respectively fixedly connected to inner sides of the two second casing components.

4. The migraine treatment device of claim 1, wherein a main mask is placed at an outer side of a front end of the therapeutic device main body.

5. The migraine treatment device of claim 1, wherein a microphone and a speaker are placed inside the therapeutic device main body, and a signal receiving hole, a charging port, and a button are placed on an outer wall of the therapeutic device main body.

6. The migraine treatment device of claim 1, wherein a first foam component, a second foam component, a third foam component, and an elastic fabric are successively placed on an inner side of an overall casing of the therapeutic device main body, and a female buckle is placed on an outer side of the elastic fabric, a plurality of through holes are provided on the second foam component, and a vibration motor is placed in each through hole.

7. The migraine treatment device of claim 6, further comprising a heating device and a thermistor that is adapted to the heating device, wherein the heating device and the thermistor are placed inside the therapeutic device main body, and the heating device is located between the second foam component and the third foam component.

8. The migraine treatment device of claim 1, wherein straps and adjustment devices that are adapted to the straps are placed on both sides of the therapeutic device main body.

9. The migraine treatment device of claim 1, wherein the therapeutic device main body has a structure of an eye mask.

10. A method for controlling a migraine treatment device, comprising:
providing a therapeutic device main body, wherein a fixed therapeutic electrode pad is placed on an upper inner side of the therapeutic device main body;
providing a green light emitting board placed inside the therapeutic device main body, wherein green light emitting diode (LED) lights are evenly arranged on both sides of the green light emitting board;
providing a light transmitting component placed inside the therapeutic device main body, wherein the light transmitting component is adapted to the green light emitting board, and
providing a lithium battery that supplies power to the therapeutic device main body.

11. The method of claim 10, further comprising:
providing a movable therapeutic electrode pad and an ear clip wire, wherein an electrode connection wire is connected to the movable therapeutic electrode pad, and an output interface that is adapted to the electrode connection wire and the ear clip wire is placed outside the therapeutic device main body.

12. The method of claim 10, further comprising:
providing the therapeutic device main body with a foldable structure, and
providing a first casing component, two second casing components, and two third casing components that are sequentially arranged from an outer side to an inner side as an overall casing of the therapeutic device main body, wherein the two second casing components are respectively pivotally connected to two sides of the first casing component, and the two third casing components are respectively fixedly connected to inner sides of the two second casing components.

13. The method of claim 10, further comprising:
placing a main mask at an outer side of a front end of the therapeutic device main body.

14. The method of claim 10, further comprising:
placing a microphone and a speaker inside the therapeutic device main body, and
placing a signal receiving hole, a charging port, and a button on an outer wall of the therapeutic device main body.

15. The method of claim 10, further comprising:
successively placing a first foam component, a second foam component, a third foam component, and an elastic fabric on an inner side of an overall casing of the therapeutic device main body, and
placing a female buckle on an outer side of the elastic fabric, and
providing a plurality of through holes on the second foam component, and
placing a vibration motor in each through hole.

16. The method of claim 15, further comprising:
placing the heating device and the thermistor are placed inside the therapeutic device main body, and
placing the heating device between the second foam component and the third foam component.

17. The method of claim 10, further comprising:
placing straps and adjustment devices that are adapted to the straps on both sides of the therapeutic device main body.

18. The method of claim 10, wherein the therapeutic device main body has a structure of an eye mask.
